(19) United States
(12) Patent Application Publication
(10) Pub. No.: US 2007/0197681 A1
(43) Pub. Date: Aug. 23, 2007

(54) Lens surface enhancement

(75) Inventors: Michael D. Lowery, Vista, CA (US); Laurent Hoffmann, Aliso Viejo, CA (US)

Correspondence Address: ADVANCED MEDICAL OPTICS, INC., 1700 E. ST. ANDREW PLACE, SANTA ANA, CA, 92705, US

(73) Assignee: Advanced Medical Optics, Santa Ana, CA (US)

(21) Appl. No.: 11/360,237

(22) Filed: Feb. 22, 2006

(51) Int. Cl. A61F 2/16

(52) U.S. Cl. 523/1; 623/6.62

(57) ABSTRACT

An intraocular lens with a hydrophilic polymer coating composition and methods for using same are provided. Specifically, a composition suitable for reducing tackiness in intraocular lens is provided wherein an acrylic intraocular lens is treated by vapor deposition with an alkoxy silyl terminated polyethylene glycol polymer composition. Methods for making an intraocular lens with a hydrophilic polymer coating are also provided.

### FIELD OF THE INVENTION

The present invention relates to intraocular lens coating compositions and particularly to polyethylene glycol coatings to decrease the tackiness of soft acrylic intraocular lenses.

### BACKGROUND OF THE INVENTION

The human eye is a highly evolved and complex sensory organ. It is composed of a cornea, or clear outer tissue which refracts light rays en route to the pupil, an iris which controls the size of the pupil thus regulating the amount of light entering the eye, and a lens which focuses the incoming light through the vitreous fluid to the retina. The retina converts the incoming light into electrical energy that is transmitted through the brain stem to the occipital cortex resulting in a visual image. In the perfect eye the light path from the cornea, through the lens and vitreous fluid to the retina is unobstructed. Any obstruction or loss in clarity within these structures causes scattering or absorption of light rays resulting in diminished visual acuity. For example, the cornea can become damaged resulting in edema, scarring or abrasions, the lens is susceptible to oxidative damage, trauma and infection, and the vitreous can become cloudy due to hemorrhage or inflammation.

As the body ages, the effects of oxidative damage caused by environmental exposure and endogenous free radical production accumulate resulting in a loss of lens flexibility and denatured proteins that slowly coagulate, reducing lens transparency. The natural flexibility of the lens is essential for focusing light onto the retina by a process referred to as accommodation. Accommodation allows the eye to automatically adjust the field of vision for objects at different distances. A common condition known as presbyopia results when the cumulative effects of oxidative damage diminish this flexibility reducing near vision acuity. Presbyopia usually begins to occur in adults during their mid-forties; mild forms are treated with glasses or contact lenses.

Lenticular cataracts are a lens disorder resulting from protein coagulation and calcification. There are four common types of cataracts: senile cataracts associated with aging and oxidative stress, traumatic cataracts which develop after a foreign body enters the lens capsule or following intense exposure to ionizing radiation or infrared rays, complicated cataracts which are secondary to diseases such as diabetes mellitus or eye disorders such as detached retinas, glaucoma and retinitis pigmentosa, and toxic cataracts resulting from medicinal or chemical toxicity. Regardless of the cause, the disease results in impaired vision and may lead to blindness.

Treatment of severe lens disease requires the lens' surgical removal or phacoemulsion followed by irrigation and aspiration. However, without a lens the eye is unable to focus the incoming light on the retina. Consequently, artificial lenses must be used to restore vision. Three types of prosthetic lenses are available: cataract glasses, external contact lenses and intraocular lenses (IOL). Cataract glasses have thick lenses, are uncomfortably heavy and cause vision artifacts such as central image magnification and side vision distortion. Contact lenses resolve many of the problems associated with glasses, but require frequent cleaning, are difficult to handle (especially for elderly patients with symptoms of arthritis) and are not suited for persons who have restricted tear production. Intraocular lenses are used in the majority of cases to overcome the aforementioned difficulties associated with cataract glasses and contact lenses.

Intraocular lenses were first used as a replacement for damaged natural crystalline lenses in 1949. These early IOL experiments were conducted in England by Dr. Howard Ridley, an RAF ophthalmologist. Dr Ridley first observed acrylate polymer biocompatibility in the eyes of pilots who had sustained ocular injuries from polymethylmethacrylate (PMMA) shards when their aircraft canopies were shattered. However, it took nearly thirty years for ophthalmologists to embrace IOL implantation as a routine method for restoring vision in patients suffering from diseased or damaged natural crystalline lenses.

There are four primary IOL categories: non-deformable, foldable, expansible hydrogels and injectable. Early non-deformable IOL implants were ridged structures composed of acrylates and methacrylates requiring a large incision in the capsular sac and were not accommodative. This large incision resulted in protracted recovery times and considerable discomfort for the patient. In an effort to reduce recovery time and patient discomfort numerous small incision techniques and lenses have been developed.

Early IOLs were made from PMMA because of its proven biocompatibility. Polymethylmethacrylate is a rigid polymer and requires a 5 mm to 7 mm incision. Incision size is directly related to patient trauma, discomfort and healing times. Moreover, incisions sizes in the 5 mm to 7 mm range generally require sutures further increasing procedural complexity and patent discomfort. Lens size dictates incision size and lens size is in turn determined by the size of the capsular sac and natural crystalline lens. Thus lenses made from a rigid polymer such as PMMA require an incision size at least as large as the minimum IOL dimension which is generally 5.5 mm on average.

In an effort to decrease incision size and corresponding patient discomfort, recovery time and procedural complexity, a number of IOL designs suitable for insertion through small incisions have been developed; most notably foldable IOLs. Foldable IOLs are made from non-rigid, or flexible polymers including hydrophobic acrylics, hydrophilic hydrogels, silicone elastomers and porcine collagen. Intraocular lenses made from these materials can be folded or rolled into implantable configurations having minimum dimensions suited for 3 mm incisions, or less. The folded IOL is inserted through a small incision and the IOL then unfolds slowly and gently as it warms within the capsular bag. The IOLs also often have at least one haptic for fixation in the posterior or anterior chamber of the eye.

However, foldable acrylic IOLs have an inherent tackiness and can make implantation more difficult and damage ocular tissues. Therefore there exists a need for a non-tacky foldable soft acrylic IOL.

### SUMMARY OF THE INVENTION

The present invention provides intraocular lenses (IOL) with coatings suitable for reducing tackiness in the lens and methods for providing IOLs with the coatings. More specifically, the present invention provides coated IOLs comprising an acrylic polymer substrate and a polyethylene glycol coating material for making the IOL less tacky and thereby reducing the risk of damage to the lens either before or during insertion.

In one embodiment of the present invention, an intraocular lens is provided having a non-tack coating comprising a polyethylene glycol polymer having a plurality of monomers of the structure of Formula 1: 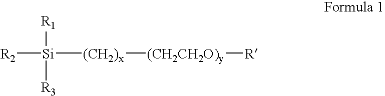
wherein R₁, R₂ and R₃ can be, individually or a halogen or alkoxy group, x is an integer between 2 and 5, y is an integer between 5 and 15, and R′ is a non-reactive group. In an embodiment, the halogen is selected from the group consisting of Cl, Br and I. In another embodiment, the alkoxy is methoxy or ethoxy. In yet another embodiment, R₁, R₂ and R₃ all comprise methoxy groups. In an embodiment, x is an integer between 2 and 5 and y is an integer between 5 and 15. In another embodiment, the non-reactive group is a low molecular weight alkyl group. In yet another embodiment, the low molecular weight alkyl group is methyl.

In another embodiment of the present invention, an IOL is provided having a non-tack coating comprising a polyethylene glycol polymer having a plurality of monomers wherein the monomer has the structure of Formula 2.

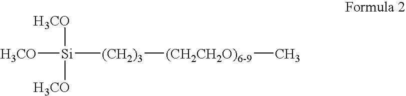

In one embodiment of the present invention, a method for providing an intraocular lens surface with a hydrophilic polymer coating is provided comprising: applying at least one hydrophilic polymer coating to at least one surface of the intraocular lens using vapor deposition.

In another embodiment of the methods of the present invention, the at least one hydrophilic polymer coating is comprised of monomers having the structure of Formula 1: 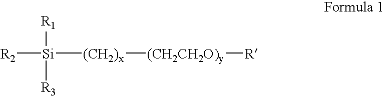
wherein R₁, R₂ and R₃ can be, individually or a halogen or alkoxy group, x is an integer between 2 and 5, y is an integer between 5 and 15, and R′ is a non-reactive group. In an embodiment, the halogen is selected from the group consisting of Cl, Br and I. In another embodiment, the alkoxy is methoxy or ethoxy. In yet another embodiment, R₁, R₂ and R₃ all comprise methoxy groups. In an embodiment, x is an integer between 2 and 5 and y is an integer between 5 and 15. In another embodiment, the non-reactive group is a low molecular weight alkyl group. In yet another embodiment, the low molecular weight alkyl group is methyl.

In another embodiment of the methods present invention, a method for providing an intraocular lens surface with a hydrophilic polymer coating, wherein the hydrophilic polymer coating is comprised of polymers and the monomer has the structure of Formula 2.

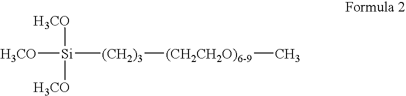

### BRIEF DESCRIPTION OF THE DRAWINGS

FIG. 1 depicts a process flow chart for molecular vapor deposition of PEG coatings on intraocular lenses according-to the teachings of the present invention.

### DETAILED DESCRIPTION OF THE INVENTION

The present invention provides intraocular lenses with coatings suitable for reducing tackiness. More specifically, the present invention provides coated intraocular lenses comprising an acrylic polymer substrate and a polyethylene glycol (PEG) coating material. Coating the surface of soft acrylic IOLs according to the teachings of the present invention acts to reduce cell and tissue adhesion as well as decrease tackiness of the IOL to itself and to surgical instruments. This tackiness increases the risk that the IOL will be marred or damaged prior to or during implantation.

Polyethylene glycol is a neutral hydrophobic polymer having good blood and tissue compatibility. In one embodiment of the present invention, a trialkoxy silyl terminated PEG coating, made according to the teachings of the present invention, is highly effective in reducing the self-tack of acrylic IOLs. This coating allows the IOL to smoothly unfold during the insertion process with minimal tendency for the leading haptic to adhere to the optic body or the IOL to adhere to itself or the insertion apparatus.

Hydrophilic polymers suitable for use in the IOL coating of the present invention include monomeric precursor units of Formula 1: 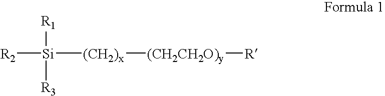
wherein R₁, R₂ and R₃ can be, individually or a halogen including, but not limited to Cl, Br or I, or alkoxy group including, but not limited to methoxy and ethoxy; x is an integer between 2 and 5; y is an integer between 5 and 15; and R′ is a non-reactive group such as, but not limited to a low molecular weight alkyl group such as methyl.

In one embodiment of the present invention, a preferred monomeric precursor unit suitable for use in the hydrophilic polymer coating of the present invention is 2-[methoxy(polyethyleneoxy)propyl]trimethoxysilane (CAS No. 65994-07-2, Gelest, Inc.), the monomer precursor unit of Formula 2:

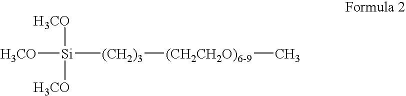

The PEG coating compositions of the present invention are applied to an IOL substrate in the form of a monolayer. In an exemplary embodiment, the coating of the present invention is applied using vapor deposition, including physical deposition and chemical deposition. An exemplary form of vapor deposition is the Molecular Vapor Deposition (MVD™) method of Applied Microstructures Inc. (San Jose, Calif.). The MVD™ method is disclosed in U.S. Patent Application Publication Serial Numbers US2005/0271809, US2005/0271810, US2005/0271893, and US2005/0271900, the contents of which are incorporated by reference herein for all they contain regarding molecular vapor deposition.

In addition to PEG silanes, other volatile organosilanes find utility as surface active agents. Examples include: n-hexadecyltrichlorosilane C16H33Cl3Si (Gelest SIH5920.0); hexadecyltriethoxysilane, C₂₂H₄₈O₃Si (Gelest SIH5922.0); hexadecyltrimethoxysilane C₁₉H₄₂O₃Si (Gelest SIH5925.0); (heptadecafluoro-1,1,2,2-tetrahydrodecyl)trimethoxysilane, C₁₃H₁₃F₁₇O₃Si (Gelest, SIH5841.5); (heptadecafluoro-1,1,2,2-tetrahydrodecyl)trichlorosilane, C₁₀H₄,Cl₃,F₁₇Si (Gelest SIH5841.0); and (heptadecafluoro-1,1,2,2-tetrahydrodecyl)triethoxysilane, C₁₆H₁₉F₁₇O₃Si (Gelest SIH5841.2). These examples are illustrative of the potential of the method and should not be considered limiting in their scope.

The PEG coating composition of the present invention is applied to an IOL substrate in need of coating to reduce tackiness. The IOL substrate may be comprised of any opthalmically acceptable material, such as silicone, hydrogels or hydrophobic acrylic materials. A preferred intraocular lens substrate is an acrylic polymer material.

An IOL substrate suitable for coating with the PEG coating of the present invention is formed from a hydrophobic deformable-elastic transparent cross-linked acrylic material with a unique balance of flexibility, elasticity, tensile strength and softness properties yielding significant advantages during implantation and subsequent use. More specifically, because of its improved flexibility, the IOL is capable of being reduced in profile size to fit through an incision of reduced size in comparison to conventional hard plastic lenses composed of polymethylmethacrylate (PMMA) or the like. Because of its controlled elasticity, the lens body anchors the haptics with sufficient damping to prevent snap-action movement of the haptics toward their normal unstressed configurations, thereby preventing the haptics from sharply striking and damaging eye tissue. Moreover, the lens body possesses a relatively slow speed of return or retraction of about 20-180 seconds from a deformed rolled shape to its initial undeformed state to avoid striking and damaging eye tissue. Further, the lens body has excellent elastic memory to insure substantially complete return to the underformed state without plastic deformation in the form of fold lines or creases or other distortions which would otherwise impair optical quality.

Exemplary cross-linked acrylic materials for the coated IOLs of the present invention comprise copolymers of methacrylate and acrylate esters which are relatively hard and relatively soft at body temperature, chemically cross-linked with a diacrylate ester and cured. The resulting acrylic has a relatively leathery characteristic at temperature conditions corresponding with or approximating body temperature. More specifically, the cross-linked acrylic composition is selected to have a glass transition temperature somewhat below body temperature so that the lens will exhibit a stiffness (Young's modulus) at a body temperature environment reflecting a relatively leathery characteristic. In addition, the cross-linked acrylic composition is chosen to have highly elastic or viscoelastic properties with substantially no plastic deformation and a relatively slow speed of retraction.

With such a combination of characteristics, the IOL can be deformed as by rolling upon itself together with the haptics for facilitated implantation via a small insertion tube passed through a small incision formed in the ocular tissue at a position removed from a normal site line passing through the transparent cornea and further through the pupil for implantation through the pupil into the posterior chamber behind the iris, typically within a capsular bag which has been anteriorly ruptured in the course of extracapsular extrusion of the natural crystalline lens. The insertion tube can optionally be pre-lubricated with a lubricious material for lubrication purposes prior to inserting the IOL. Additionally, the IOL, including the lens body and haptics may be temperature prepared in advance to be substantially at body temperature, at which time the IOL and insertion tube are advanced into the eye where the lens is expelled from the tube into the eye. The thus-released lens is allowed to return to its initial nondeformed state slowly over a time of at least approximately 20 seconds. When the lens is substantially completed expanded, the lens position within the eye can be manipulated with appropriate instruments, engaging, for example positioning holes in the haptics after which the incision is closed to complete the procedure.

Table 1 lists monomers useful in preparing acrylic IOLs suitably for coating with the hydrophilic polymer coating of the present invention as well as the concentration ranges for such monomers in percent by weight and an exemplary preferred composition in percent by weight.

TABLE 1

| | Concentration | Preferred | | |
|---|---|---|---|---|
| Material | Range (wt %) | (wt %) | Tg (K) | Tg (C.) |
| Ethyl acrylate | 30-60 | 57.11 | 249 | −24 |
| Ethyl methacrylate | 25-45 | 27.71 | 339 | 65 |
| Trifluoroethyl | 5-25 | 9.82 | 355 | 82 |
| methacrylate | | | | |
| n-Butyl acrylate | 30-60 | 0 | | |
| 2-Ethyl hexyl acrylate | 30-60 | 0 | | |
| Ethyleneglycol | 0.5-4.0 | 3.75 | | |
| dimethacrylate | | | | |
| UV blocker | 0-10 | 1.5 | | |
| USP 245**, thermal | 0.05-0.2 | 0.11 | | |
| initiator | | | | |
| Total | | 100% | | |

The IOL substrates optionally further include one or more compounds selected from the group consisting of ultraviolet (UV) light absorbers and blue-violet light absorbing compounds. Ultraviolet light absorbing compounds can be any compound which absorbs light having a wavelength shorter than about 400 nm, but does not absorb any substantial amount of visible light. Suitable UV light absorbing compounds can be found in U.S. Pat. Nos. 5,164,462 and 5,217,490, the entire contents of which are hereby incorporated by reference. Non-limiting examples of UV light absorbing molecules include 2-(3′,5′-ditertiary butyl-2′-hydroxy phenyl) benzotriazole, 2-(3′-tertiary-butyl-5′-methyl-2′-hydroxy phenyl-5-chloro)benzotriazole and 2-(2′-hydroxy-5′methylphenyl)benzotriazole

In the formulation and production of the lenses of this invention, the amount of the UV absorbing molecule will be sufficient to absorb at least 90% of the ultraviolet radiation of sunlight in the 300-380 nm range but will not prevent the lens from being transparent to a substantial part of the visible spectrum.

### EXAMPLES

### Example 1

PEG Surface Treatment Procedure

Intraocular lenses suitable for coating with the PEG surface treatment of the present invention include IOLs made from acrylic polymer substrates and IOLs made of other suitable materials as are known by persons skilled in the art.

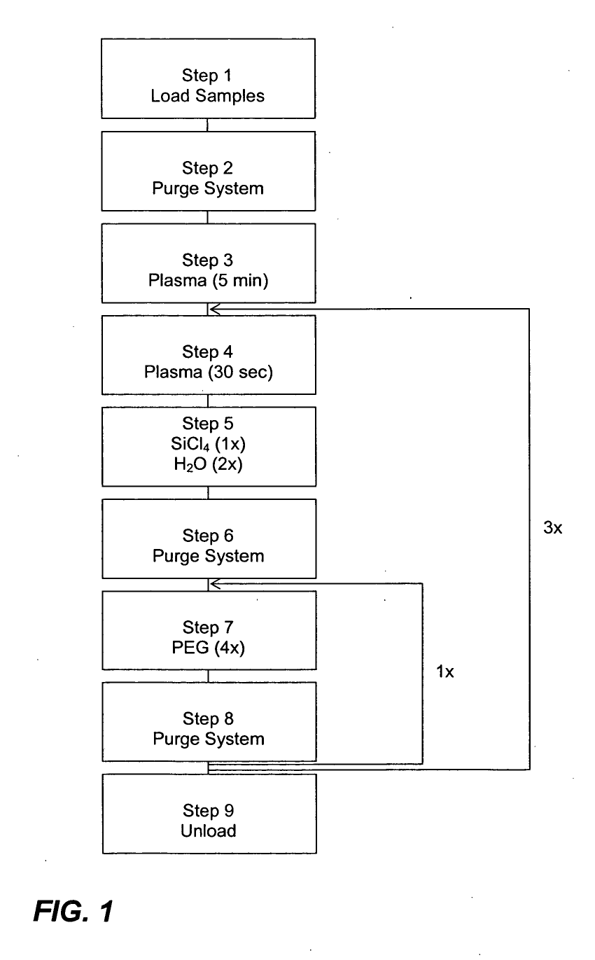

Substrates for PEG surface treatment included intraocular lenses and discs having dimensions of approximately 16.0 mm×1.0 mm. The PEG surface treatment was applied with a MVD 100 Molecular Vapor Deposition (MVD™) apparatus developed by Applied Microstructures Inc. (San Jose, Calif.). An illustrative example of the PEG treatment conditions are given in FIG. 1. Experimental conditions can be adjusted to increase or decrease the deposition of PEG.

A description of each of the process steps is given below.

Step 1: Samples are loaded onto stainless steel trays to secure the IOLs such that both of the optic surfaces are exposed to the PEG treatment. Each tray is capable of holding approximately 180 IOLs. The fixture is loaded into the MVD™ chamber. The chamber temperature is maintained at 35±1° C.

Step 2: After loading the samples, the chamber is purged to remove trace moisture and atmospheric gasses. The chamber pressure is reduced to 0.035±0.010 torr. After the desired system pressure is attained, the vacuum is discontinued and the pressure returned to ambient by filling with high purity nitrogen (N₂) gas. The vacuum/nitrogen purge cycle is repeated 5 times. At the conclusion of the purge step, the chamber is left evacuated.

Step 3: An oxygen plasma is used to clean the IOL surface and the chamber. Plasma conditions entered into the MVD™ apparatus are: oxygen (O2) flow rate 150 sccm; radio frequency power 200 wafts, duration of 5 minutes. The oxygen plasma is generated remote from the reaction chamber.

Step 4: The process flow diagram now enters the main processing loop. The cycle begins with a brief oxygen plasma exposure. Plasma conditions are: O₂ flow rate 150 sccm; radio frequency power 200 watts, duration of 30 seconds.

Step 5: A SiO₂ coating is formed on the IOL surface. High purity silicone tetrachloride (Gelest, SIT7085.0) and sterile water (Baxter) are introduced into the reaction chamber. The chamber pressures are: after SiCl₄ injection 1.30 torr, after first water addition 1.90 torr, after second water injection 2.70 torr. The chemicals are allowed to react for 10 minutes.

Step 6: The chamber is purged with five (5) nitrogen flushes as described in Step 2. This step insures that any excess reagents are removed prior to the introduction of the PEG silane.

Step 7: Methoxy(polyethyleneoxy)propyltrimethoxysilane (Gelest, SIM6492.7) is introduced into the reaction chamber. Four injections having a line pressure of 0.50 torr are used. After the PEG injections, the reaction is allowed to continue for 15 minutes.

Step 8: The chamber is purged with five (5) nitrogen flushes as described in Step 2. This step insures that any excess reagents are removed from the chamber. Steps 7 and 8 are repeated an additional one (1) time as shown in the diagram.

Steps 4-8 are repeated a total of three (3) times.

Step 9: The system is filled with nitrogen to ambient pressure and the IOLs removed.

After the PEG treatment, the acrylic IOLs and/or discs are characterized for effectiveness of the deposition process. The treatment process is intended to introduce sufficient PEG onto the lens surface to reduce the material self-tack and allow for controlled, rapid lens unfolding (unfold time<1 minute). The treatment must be thin enough not affect the optical characteristics of the lens.

The PEG surface treatment was evaluated using contact angle goniometry, attenuated total reflectance Fourier transform infrared spectroscopy (ATR-FTIR), angle resolved X-ray photoelectron spectroscopy (XPS). The effectiveness of the treatment for tack reduction was measured by measuring the time required for the IOL to unfold post when subjected to simulated use. The depth of the SiO₂ layer was estimated based on measurements made on silicone wafers (profilemetry).

| | | Number | | |
|---|---|---|---|---|
| Experiment | SiO₂ Treatment | of PEG | Contact Angle | Surface |
| Number | Thickness (A) | Cycles | (degrees) | Appearance |
| 1 | None | 8 | 38 | OK |
| 2 | 20 | 4 | 26 | OK |
| 3 | 20 | 8 | 31 | OK |
| 4 | 60 | 4 | 23 | Lt. Reflections |
| 5 | 400 | None | <5 | Crazing |
| 6 | None | 4 | 29 | OK |
| 7 | None | 2 | 32 | OK |
| 8 | 20 | None | 31 | OK |
| 9 | 20 | 2 | 29 | OK |
| 10 | 150 | None | <5 | Reflections |
| 11 | 60 | 2 | 20 | OK |
| 12 | 60 | 8 | 28 | OK |
| 13 | None | None | 33 | OK |
| 14 | 150 | 4 | 19 | Crazing |
| 15 | 60 | None | 17 | OK |
| 16 | 150 | 4 | 16 | OK |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An intraocular lens having a non-tack coating comprising a polyethylene glycol polymer having a plurality of monomers of the structure of Formula 1: 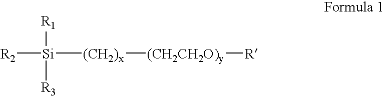
wherein R₁, R₂ and R₃ can be, individually or a halogen or alkoxy group;
x is an integer between 2 and 5;
y is an integer between 5 and 15; and
R′ is a non-reactive group.

2. The intraocular lens of claim 1 wherein said halogen is selected from the group consisting of Cl, Br and I.

3. The intraocular lens of claim 1 wherein said alkoxy group is methoxy or ethoxy.

4. The intraocular lens of claim 3 wherein R₁, R₂ and R₃ all comprise methoxy groups.

5. The intraocular lens of claim 1 wherein x is an integer between 2 and 5.

6. The intraocular lens of claim 1 wherein y is an integer between 5 and 15.

7. The intraocular lens of claim 1 wherein said non-reactive group is a low molecular weight alkyl group.

8. The intraocular lens of claim 7 wherein said low molecular weight alkyl group is methyl.

9. The intraocular lens of claim 1 wherein said monomer has the structure of Formula 2:

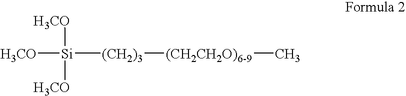

10. A method for providing an intraocular lens surface with a hydrophilic polymer coating comprising:
applying at least one hydrophilic polymer coating to at least one surface of said intraocular lens using vapor deposition.

11. The method according to claim 10 wherein said at least one hydrophilic polymer coating is comprised of monomers having the structure of Formula 1: 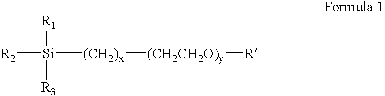
wherein R₁, R₂ and R₃ can be, individually or a halogen or alkoxy group;
x is an integer between 2 and 5;
y is an integer between 5 and 15; and
R′ is a non-reactive group.

12. The method according to claim 11 wherein said halogen is selected from the group consisting of Cl, Br and I.

13. The method according to claim 11 wherein said alkoxy group is methoxy or ethoxy.

14. The method according to claim 13 wherein R₁, R₂ and R₃ all comprise methoxy groups.

15. The method according to claim 11 wherein x is an integer between 2 and 5.

16. The method according to claim 11 wherein y is an integer between 5 and 15.

17. The method according to claim 11 wherein said non-reactive group is a low molecular weight alkyl group.

18. The method according to claim 17 wherein said low molecular weight alkyl group is methyl.

19. The method according to claim 11 wherein said monomer has the structure of Formula 2: [chemical structure]
